(19) United States
(12) Patent Application Publication
(10) Pub. No.: US 2023/0241188 A1
(43) Pub. Date: Aug. 3, 2023

(54) PREVENTING AND TREATING VIRAL INFECTIONS

(71) Applicant: Mucpharm Pty Ltd, Victoria (AU)

(72) Inventors: David MORRIS, New South Wales (AU); Sarah Valle, New South Wales (AU)

(73) Assignee: Mucpharm Pty Ltd, Victoria (AU)

(21) Appl. No.: 17/922,709

(22) PCT Filed: Apr. 30, 2021
(86) PCT No.: PCT/AU2021/050401

§ 371 Date: Nov. 1, 2022

(30) Foreign Application Priority Data
May 1, 2020 (AU) 2020901397

(51) Int. Cl. A61K 38/48; A61K 31/195; A61P 31/14; A61K 9/00; A61K 45/06

(52) U.S. Cl. CPC A61K 38/4873; A61K 9/0078; A61K 31/195; A61K 45/06; A61P 31/14

(57) ABSTRACT

Disclosed herein is a method for the prophylaxis or treatment of a viral infection in a patient. The method comprises administering to the patient a therapeutically effective amount of a combination of a glycoprotein affecting protease and a disulphide bond breaking agent.

### TECHNICAL FIELD

The present invention relates to methods for the prophylaxis or treatment of viral infections in a patient. The present invention also relates to methods for rendering virus non-infective.

### BACKGROUND ART

Viral infections are a significant cause of illness and death of humans and other animals. Given the large number and wide variety of viruses, as well as their ability to mutate, the development of methods for the prophylaxis and treatment of viral infections has been an enduring challenge.

In many cases, treatment of viral infections focuses on symptomatic relief and not fighting the virus. Some antiviral medications, however, work directly on viruses, generally by inhibiting their reproduction via many different mechanisms. Successful antiviral medications have, for example, used fusion inhibitors to prevent the virus from fusing with the host cell or used antiproteases to target viral proteases essential for reproduction.

The pandemic caused by the severe acute respiratory syndrome coronavirus 2 (SARS-CoV2) has provided an urgency without precedent in the modern world to discover and develop novel methods for prophylaxis and treatment of viral infections.

### SUMMARY OF INVENTION

In a first aspect, the present invention provides a method for the prophylaxis or treatment of a viral infection in a patient. The method comprises administering to the patient a therapeutically effective amount of a combination of a glycoprotein affecting protease and a disulphide bond breaking agent.

As will be described in further detail below, the present inventors have discovered that the combination of a specific glycoprotein affecting protease and disulphide bond breaking agent is effective to disintegrate proteins found on the surfaces of some viruses. As these proteins are likely to play a crucial role in the mechanism via which the virus internalises within host cells, the inventors believed that the results of their preliminary experiments lead to a reasonable prediction of the therapeutic applications disclosed herein. Subsequent experiments (also described below) have found that this combination was effective to prevent infection of some cell lines. Further experiments, both currently underway and planned, will confirm the inventors’ prediction.

Given that proteases are essential for the reproduction of many viruses, and that these proteases are a recognised target for some antiviral medications, it was surprising to the inventors that a potential antiviral medication might involve the use of a protease such as a glycoprotein affecting protease.

In some embodiments, the glycoprotein affecting protease may be a cysteine protease, for example bromelain. Advantages of using bromelain will be described below.

In some embodiments, the disulphide bond breaking agent may be acetylcysteine (NAC). Advantages of using NAC will also be described below.

In some embodiments, the combination may be administered into the lungs of the patient. The combination may, for example, be nebulized before administration.

In some embodiments, the combination may be nasally administered to the patient.

In some embodiments, the combination may be intravenously administered to the patient.

In some embodiments, the combination may be administered to the patient immediately upon the patient becoming symptomatic. As will be described below, the inventors believe that early treatment, especially if the composition is delivered to the areas of the body (e.g. the nasal cavity) where the virus is likely to initially infect, may help to prevent (or at least ameliorate) subsequent infection in the patient’s lungs. The inventors’ data shows promise at early stages of infection as being effective for preventing disease progression.

In some embodiments, the combination may be administered to the patient as a prophylactic, that is, when there is a concern that the patient may be imminently exposed to the virus.

In some embodiments, one or more additional therapeutic agents may be co-administered to the patient with the combination. Such additional therapeutic agents may, for example be selected from the group consisting of antivirals, antibacterial agents and antiproteases.

In some embodiments, the glycoprotein affecting protease, disulphide bond breaking agent and, optionally, any other additional therapeutic agent(s), may be administered to the patient simultaneously, separately or sequentially.

In some embodiments, the viral infection may be a viral respiratory disease such as COVID-19, the disease caused by severe acute respiratory syndrome coronavirus 2 (SARS-CoV-2). In some embodiments, the viral infection may be a viral haemorrhagic fever such as an ebolavirus.

In a second aspect, the present invention provides a method for rendering a virus non-infective. The method comprises contacting the virus with a combination of a glycoprotein affecting protease and a disulphide bond breaking agent.

In some embodiments, the virus may be a human coronavirus such as severe acute respiratory syndrome coronavirus 2 (SARS-CoV-2). In some embodiments, the virus may be an ebolavirus.

In some embodiments of the method of the second aspect, the virus may be contacted with the combination of the glycoprotein affecting protease and disulphide bond breaking agent by spraying the combination on to the virus (e.g. using a nasal spray, throat spray or intra-tracheal spray).

In some embodiments, the combination may be sprayed into the patient immediately upon the patient becoming symptomatic, for the reasons described above. In some embodiments, the combination may be sprayed into the patient pre-emptively for a prophylactic effect.

In a third aspect, the present invention provides the use of a combination of a glycoprotein affecting protease and a disulphide bond breaking agent as an antiviral agent.

In a fourth aspect, the present invention provides the use of a combination of a glycoprotein affecting protease and a disulphide bond breaking agent for the prophylaxis or treatment of a viral infection in a patient.

In a fifth aspect, the present invention provides the use of a combination of a glycoprotein affecting protease and a disulphide bond breaking agent for the preparation of a medicament for the prophylaxis or treatment of a viral infection in a patient.

In a sixth aspect, the present invention provides the use of a combination of a glycoprotein affecting protease and a disulphide bond breaking agent for rendering a virus non-infective or non-viable.

In a seventh aspect, the present invention provides a combination of a glycoprotein affecting protease and a disulphide bond breaking agent for use in the prophylaxis or treatment of a viral infection in a patient.

In an eight aspect, the present invention provides a method for preventing disease progression in a patient infected by a virus, the method comprising administering to the patient a therapeutically effective amount of a combination of a glycoprotein affecting protease and a disulphide bond breaking agent.

Specific features of embodiments of the third to eighth aspects of the present invention may be as described herein in greater detail with reference to the first and second aspects.

Other aspects, features and advantages of the present invention will be described below.

### BRIEF DESCRIPTION OF THE DRAWING

FIG. 1 is a photograph of an SDS-PAGE gel after electrophoresis had been carried out with samples containing SARS-CoV-2 (2019-nCoV) Spike S1+S2 ECD-His Recombinant Protein incubated with different concentrations of bromelain and/or NAC for 30 mins at 37° C.;

FIG. 2 is a photograph of an SDS-PAGE gel after electrophoresis had been carried out with samples containing SARS-CoV-2 (2019-nCoV) Envelope Recombinant Protein incubated with different concentrations of bromelain and/or NAC for 30 mins at 37° C.;

FIG. 3 is a graph showing the results of a differential assay between NAC and DTT for the reduction of disulphide bonds found in spike (B) and envelope (C) protein;

FIG. 4 shows graphs showing the cytopathic effect ratio by dilutions of BromAc with Bromelain at varying concentrations and Acetylcysteine 20 mg/ml on SARS-CoV-2 in Vero cells (A) and BGM cells (B);

FIG. 5 shows graphs showing the impact of Bromelain and Acetylcysteine treatment on SARS-CoV-2 cytopathic effect and level of replication when cultured in-vitro at different dilutions in Vero cells;

FIG. 6 shows graphs of optical density (OD) measured by cell staining with Neutral Red, where optical density (OD) is directly proportional to cell viability of wild-type (WT)SARS-CoV-2 strain (A and B) and spike mutant (ΔS) SARS-CoV-2 strain (A and B) upon treatment with Bromelain, Acetylcysteine and BromAc;

FIG. 7 shows a threshold matrix of log₁₀ reduction values (LRV) of in vitro virus replication 96 h after BromAc treatment on WT and ΔS SARS-CoV-2 strains at 5.5 and 4.5 log₁₀TCID₅₀/mL titers;

FIG. 8 shows a graph of SARS-CoV-2 replication capacity of WT and ΔS SARS-CoV-2 measured by Real-Time Cell Analysis;

FIG. 9 show western blot analysis results for the treatment of VERO cells and MDA-MB-231 cells with Bromelain, Acetylcysteine and BromAc;

FIG. 10 shows photographs of SDS-PAGE gels after electrophoresis had been carried out with samples containing ebolavirus spike recombinant proteins incubated with different concentrations of bromelain and/or NAC for 30 mins at 37° C.; and

FIG. 11 is a graph showing the percentage of body weight fluctuation of mice treated with a nasal spray of BromAc.

### DETAILED DESCRIPTION OF THE INVENTION

As noted above, the present invention provides a method for the prophylaxis or treatment of a viral infection in a patient. The method comprises administering to the patient a therapeutically effective amount of a combination of a glycoprotein affecting protease and a disulphide bond breaking agent.

The present invention also provides a method for rendering a virus non-infective. This method comprises contacting the virus with a combination of a glycoprotein affecting protease and a disulphide bond breaking agent.

Many viruses have outer surfaces that include functions that enable them to bind to and subsequently internalise within host cells. The inventors believe that the discovery which resulted in the invention the subject of the present application, namely that the combinations disclosed herein disintegrate proteins found on the surfaces of some viruses and renders them non-infective, may have applicability to any virus having glycoprotein-containing functions on their surfaces.

For example, the novel SARS-CoV-2 virus with its clinical syndrome known as COVID-19, is made up of a number of glycoproteins, including spike protein (S), nucleocapsid protein (N), membrane protein (M) and envelope protein (E). The spike protein that is responsible for initiating internalisation of the virus genome into human lung cells protrudes on the outer surface, and is made up of number of amino acids and glycoproteins.

The present invention has been made following the inventors’ discovery that a combination of bromelain and acetylcysteine (NAC) caused the spike protein of severe acute respiratory syndrome coronavirus 2 (SARS-CoV-2) to disintegrate. The inventors subsequently discovered that this combination also disintegrates the envelope protein (and perhaps the membrane protein) of the virus. Further, in live virus tests (described below), the combination prevented infection in various cell lines.

Without wishing to be bound by theory, the inventors believe that the S and E proteins, which are described as being held together and structurally supported by disulphide bonds, are effectively disintegrated by the combination of bromelain and NAC. This unique combination, in the context of antiviral infections, is thought to operate via two separate mechanisms that result in a more complete disintegration of the proteins than could possibly be achieved if the agents were used separately. It is thought that the bromelain hydrolyses the glycosidic bonds of the glycoproteins in the proteins whilst, at or about the same time, the NAC breaks the disulphide bridges that may still be holding the protein in its tertiary structure. Complete denaturation ensues, resulting in the virus being non-infective. Removal of the spike protein is a different means of treatment, in comparison to many existing antiviral drugs. It is not aimed at replication but to prevent binding of the virus to the host.

The integrity of the proteins (S, N, M & E) is vital for viral functions. These proteins are formed in certain configuration for biological activities through the formation of disulphide bonds, and these bonds therefore likely play a vital role in the performance of the protein. COVID-19 also uses a glycan shield to protect it from immune recognition. Disintegrating these proteins, as the inventors’ preliminary data indicates occurs for the S protein in vitro, may result in a non-infective virus.

Another virus upon which the present invention has been tested is ebolavirus. Ebola is an extremely serious but relatively rare viral haemorrhagic fever, characterized by acute systemic manifestations with vascular damage, plasma leakage, severe inflammation, and disruption of the immune system. It spreads through patients by direct contact with body fluids from an infected person, such as cough droplets, respiratory sputum, faeces, urine, etc. An approx. 90% fatality rate has been reported although there is now an approved antibody “cocktail”, REGN-EB3, which has reportedly reduced mortality by 33-35%. Similar to SARC-CoV-2 and other virus, ebolavirus entry into host cells appears to require the surface glycoprotein to initiate attachment and fusion of viral and host membranes.

The term “BromAc”, as used herein, is a combination of the drugs Bromelain and acetylcysteine, which has been developed by some of the present inventors for treating mucinous cancers. BromAc was found to rapidly dissolve and remove tumour mucin, whilst neither of the drugs worked alone. BromAc has been shown to remove the mucin protective framework expressed by cancer including MUC1, MUC2, MUC4, MUC5B, MUC5AC and MUC16 due to its effect on glycoproteins and disulphide bonds. It also combines synergistically with a variety of anticancer drugs.

The inventors speculated that the proteins of the SARS-CoV-2 virus are all highly likely to be affected by BromAc and it is possible that BromAc will remove the many glycoproteins and potentially render the virus non-infective. The inventors’ preliminary in vitro studies have shown that BromAc destroys the SARS-CoV-2 S and E proteins. Subsequent studies (described below) have shown that BromAc destroys spike proteins on ebolavirus and the inventors believe that it is plausible that the present invention will have therapeutic effect in relation to the prophylaxis or treatment of infections caused by other viruses that use spike proteins to attach to host cells.

In addition, both agents in BromAc have mucolytic activity, which may be especially useful in treating respiratory viral infections. Indeed, oxygen exchange is one of the primary problems in patients that present with the novel coronavirus infections, where patients succumb to acute respiratory distress syndrome (ARDS) and associated diffuse alveolar disease (DAD). The development of thick mucinous sputum in patients with SARS-CoV-2 is variable at the early stages of the illness. Approx. 30-40% of patients that present to hospital with COVID-19 have sputum production. The sputum has been described as a sticky and thick mucinous material that may be brown or clear and is difficult to cough up. In a recent study on lung pathology of COVID-19 patients, there were increased levels of MUC1 and MUC5AC in the sputum aspirated from the trachea. The pathologic findings from autopsy samples indicated that the most defining characteristics of COVID-19 was mucus staggering in the bronchioles and alveoli, with photomicrographs showing a highly proteinaceous material filling the alveoli air spaces.

The inventors note that as BromAc removes a range of MUC types (as described above), and that others have shown that acetylcysteine removes MUC5AB, then it is reasonable to predict that BromAc, with its demonstrated ability to destroy the S and surface proteins (E, M) on the virus, may also rapidly dissolve and remove the proteinaceous material from the alveoli, potentially allowing improved ventilation and gas exchange and transfer.

The present invention therefore finds particular application for the prophylaxis or treatment of COVID-19, which is the disease caused by SARS-CoV-2. It is expected, however, that the present invention may be useful for the prophylaxis or treatment of many other viral infections, with particular emphasis on viral respiratory disease given the inventors’ previous studies on combinations of bromelain and NAC.

The present invention may be used to treat any suitable patient or subject. In some embodiments, the patient is a mammalian subject. Typically, the patient will be a human patient, although other subjects may benefit from the present invention. For example, the subject may be a pig, mouse, rat, dog, cat, cow, sheep, horse or any other mammal of social, economic or research importance.

The present invention involves the use of a combination of a glycoprotein affecting protease and a disulphide bond breaking agent, each of which will be described in turn below.

### Glycoprotein Affecting Proteases

Glycoprotein affecting proteases are proteolytic enzymes which cause proteolysis of glycoproteins. Given their preliminary data for bromelain, which is a protease enzyme that affects glycoproteins by hydrolysing glycosidic bonds within the glycoproteins, the inventors believe that any glycoprotein affecting protease may be used in the present invention, with routine trial and experimentation being all that would be required (in light of the teachings contained herein) in order to determine any particular glycoprotein affecting protease’s suitability. As used herein, the term “Glycoprotein affecting” is to be understood as affecting the glycoprotein in any therapeutically effective manner such as, for example, by digesting, liquefying or otherwise causing the glycoprotein to disintegrate. The glycoprotein affecting protease may, for example, be effective to disintegrate glycoproteins in the virus. The glycoprotein affecting protease may, for example, be effective to hydrolyse glycosidic bonds of glycoproteins in the virus.

The glycoprotein affecting protease may, for example, be a cysteine protease. Cysteine proteases (also known as thiol proteases) degrade proteins via a common catalytic mechanism, and are commonly sourced from fruits including the papaya, pineapple, fig and kiwifruit. Examples of cysteine proteases include bromelain, papain (extracted from papaya) and ananain, a plant cysteine protease in the papain superfamily of cysteine proteases.

There are other plant-derived proteolytic enzymes that express the same characteristics as Bromelain and the inventors expect that any plant-derived protease enzymes which have an effect on glycoproteins may be used in the present invention. Again, routine experimentation should be able to confirm the suitability of any particular plant-derived protease enzyme. In some embodiments, for example, the plant-derived protease enzymes may be selected from one or more of the group consisting of Bromelain, Papain (extracted from papaya), Ficain (extracted from figs), Actinidain (extracted from fruits including kiwifruit, pineapple, mango, banana and papaya), Zingibain (extracted from ginger) and Fastuosain (a cysteine proteinase from Bromelia fastuosa). Asparagus, mango and other kiwi fruit and papaya proteases may also be used.

It is expected that glycoprotein affecting protease enzymes obtained using genetic recombination may also be used in the present invention.

As used herein, Bromelain is to be understood to encompass one or more of the glycoprotein affecting and, optionally, otherwise therapeutically active substances present in the extract of the pineapple plant (Ananas Comosus). Bromelain is a mixture of substances (including different thiol endopeptidases and other components such as phosphatase, glucosidase, peroxidase, cellulase, esterase, and several protease inhibitors) and it may not be necessary for all of these substances to be included in the combination, provided that the fraction of the substances in the combination can at least affect the glycoproteins. The Bromelain used in the experiments described herein was commercially sourced from Enzybel Group.

### Disulphide Bond Breaking Agent

Disulphide bond breaking agents are species that break the disulphide bridges in proteins which help to define the tertiary structure of the protein.

In the proof of concept experiments conducted by the inventors, the disulphide bond breaking agent was acetylcysteine (NAC). Acetylcysteine is an antioxidant with reducing potential in biological systems. As the integrity of different proteins present in SARS-CoV-2 are dependent on disulphide bridges, the inventors postulate that their breakage by acetylcysteine will cause unfolding of these vital proteins, which may have detrimental effects on the performance of the proteins and hence leading to virus that are non-infective.

Advantageously, acetylcysteine is an approved product for paracetamol overdose where 21 g is given systemically over a 24-hour period. Acetylcysteine is also approved as a treatment for cystic fibrosis and chronic obstructive pulmonary disease, which is administered via inhalation, either 10% or 20% in 4ml up to four times daily. Thus, regulatory approvals for medicaments including acetylcysteine may be easier to obtain.

The present invention will primarily be described in the context of the disulphide bond breaking agent being acetylcysteine. A person skilled in the art would, however, appreciate that the teachings contained herein could likely be adapted, using routine trials and experiments, for any disulphide bond breaking agent. Examples of other disulphide bond breaking agents include cysteamine, glutathione, dithiothreitol, nacystelyn, mercapto-ethanesulphonate, carbocysteine, N-acystelyn, erdosteine, dornase alfa, gelsolin, thymosin P4, dextran, dithiobutylamine (DTBA) and heparin.

### Administration

The combination of the glycoprotein affecting protease and disulphide bond breaking agent may be administered to the patient in any manner that provides the intended therapeutic or prophylactic effect. The combination may, for example, be administered into the lungs of the patient (e.g. after being nebulized), and especially if the viral infection is a respiratory viral infection. Alternatively (or in addition), the composition may be sprayed into the patient’s nose or mouth, or even their trachea using more specialised medical equipment. Alternatively (or in addition), the combination may be nebulised and delivered into an atmosphere surrounding a patient such as a closed system tent or other closed-in environmental spaces for treatment.

Systemic administration (e.g. via injection or intravenously) might also be appropriate in some circumstances, depending on the nature of the virus being treated.

The combination may be nasally administered to the patient. Recent studies have suggested that the first site of infection of the SARS-Cov-2 virus is nasopharyngeal mucosa, with a secondary movement to infect lung by aspiration. Recent studies have also shown that the nose contains the highest percentage of ACE2 receptors in the human body (up to 85%), with a ratio of over 5x in the nose than in the distal respiratory tract. If the SARS-Cov-2 virus infects the cells of the respiratory tract by fusion of the spike protein with the ACE2 receptor, as appears to be the case, then it is conceivable that targeting of the spike protein will essentially disrupt its fusion and ultimately its infective potential. This data confirms the potential importance of a therapy that can be delivered locally via the nose.

Also in line with the findings of these studies, it may be beneficial to administer the combination to the patient immediately upon the patient becoming symptomatic. In this manner, the virus may be rendered inactive at an early stage of the SARS-Cov-2 infection, before it has the opportunity to move into the lungs, whereupon it becomes less accessible and thus more difficult to treat and the risk of adverse symptoms developing increases. It may even be appropriate, in some circumstances, for at risk people to administer the composition prophylactically, for example before entering a high-risk area (e.g. an ICU ward).

Any suitable apparatus and method may be used to nasally administer the combination, using existing formulations and devices.

The glycoprotein affecting protease, disulphide bond breaking agent (and any other additional therapeutic agents, as described below) may be administered to the patient simultaneously, separately or sequentially.

The relative proportions of the glycoprotein affecting protease and disulphide bond breaking agent in the combination may vary between 10 µg/mL - 500 µg/mL (e.g. between 10 µg/mL - 250 µg/mL) of the glycoprotein affecting protease and between 2% to 10% (w/v) of the disulphide bond breaking agent. Up to 200 µg/mL bromelain in a nasal spray delivered twice daily, has been found by the inventors to be safe when administered to mice (see below) and the inventors’ further experiments will test the safety of increased amounts. The inventors note that the activity of bromelain will depend on the route of administration, for example using a nose spray versus nebuliser. The inventors expect that relatively lower doses will be effective when delivered via a nose spray. To the best of the inventors’ knowledge, no one has ever nebulised or administered bromelain into the airway before and it is noted that there is no published data on using bromelain as a respiratory therapeutic.

In some embodiments, the combination of the glycoprotein affecting protease and disulphide bond breaking agent may include one or more additional therapeutic agents for coadministration to the patient.

Any therapeutic agent having an appropriate indication in the context of treating a viral infection may be co-administered to the patient. In some embodiments, the co-administered therapeutic agent may provide symptomatic relief and not fight the virus. Alternatively, the co-administered therapeutic agent may work directly on the virus, for example via another mechanism in order to provide a more effective treatment. Examples of such therapeutic agents include antivirals (e.g. Remdeisvir, favipiravir and hydroxychloroquine), antibacterial agents (dependent on the culture in the case of a secondary bacterial infection) and antiproteases (e.g. Lopinavir-Ritonavir), corticosteroids (e.g. dexamethasone) and monoclonal antibodies.

When needed (or beneficial), the quantities of such additional therapeutic agents may be determined on an as-needed basis using no more than routine trials and experimentation.

As noted above, in its second aspect, the present invention provides a method for rendering a virus non-infective, where the virus is contacted with a combination of a glycoprotein digesting protease and a disulphide bond breaking agent.

The virus may, for example, be a human coronavirus such as SARS-CoV-2 or an ebolavirus. The virus may be internal to or external to a patient.

The virus may be rendered non-infective via any suitable mechanism. For example, the contact may result in surface glycoproteins on the virus disintegrating. For example, the contact may result in spike proteins on the virus’ surface disintegrating.

Any method via which the virus may be made to make contact with the combination of the glycoprotein digesting protease and disulphide bond breaking agent is expected to be effective in inactivate the virus. For example, the combination may be delivered as an aerosol by nebulisation via a mask or via a mechanical intubation circuit. The combination may be delivered using a nasal spray, a throat spray or an intra-tracheal spray. Alternatively (or in addition), the combination may be nebulised and delivered into a closed-in environmental space for patient treatment or for environmental decontamination purposes.

For the reasons described above, in embodiments where the combination is delivered using a spray, it would ideally be sprayed into the patient immediately upon the patient becoming symptomatic.

### Pharmaceutical Compositions

The combination of glycoprotein affecting protease and disulphide bond breaking agent used in the methods of the present invention may, in some embodiments, be provided in the form of a pharmaceutical composition comprising a pharmaceutically acceptable carrier.

Such a pharmaceutically acceptable carrier will depend on the route of administration of the composition. Liquid form preparations may include solutions, suspensions and emulsions, for example water or water-propylene glycol solutions for parenteral injection, aerosols or solutions for intranasal or intratracheal delivery. Suitable pharmaceutically acceptable carriers for use in the pharmaceutical compositions of the present invention include physiologically buffered saline, dextrose solutions and Ringer’s solution, etc.

Liquid form preparations and aerosol preparations may also be useful for intranasal administration, for example. Aerosol preparations suitable for inhalation may, for example, include solutions and solids in powder form, which may be in combination with a pharmaceutically acceptable carrier, such as an inert compressed gas, e.g. nitrogen.

Pharmaceutical compositions suitable for delivery to a patient may be prepared immediately before delivery into the patient’s body, or may be prepared in advance and stored appropriately beforehand.

The pharmaceutical compositions and medicaments for use in the present invention may comprise a pharmaceutically acceptable carrier, adjuvant, excipient and/or diluent. The carriers, diluents, excipients and adjuvants must be “acceptable” in terms of being compatible with the other ingredients of the composition or medicament and the delivery method, and are generally not deleterious to the recipient thereof. Non-limiting examples of pharmaceutically acceptable carriers or diluents which might be suitable for use in some embodiments are demineralised or distilled water; saline solution; vegetable based oils such as peanut oil, safflower oil, olive oil, cottonseed oil, maize oil; sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxylpropylmethylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3- butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from about 10% to about 99.9% by weight of the composition or medicament.

It will be understood that, where appropriate, some of the components in the combination or pharmaceutical compositions used in the present invention may be provided in the form of a metabolite, pharmaceutically acceptable salt, solvate or prodrug thereof.

“Metabolites” of the various species used in the present invention refer to the intermediates and products of the metabolism of those species.

“Pharmaceutically acceptable”, such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base addition salts that retain the biological effectiveness and properties of the components and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluene sulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as for example, tetramethylammonium hydroxide. The chemical modification of a pharmaceutical compound (i.e. drug) into a salt is a technique well known to pharmaceutical chemists to obtain improved physical and chemical stability, hygroscopicity, flow ability and solubility of compounds. See, e.g., H. Ansel et. al., Pharmaceutical Dosage Forms and Drug Delivery Systems (6th Ed. 1995) at pp. 196 and 14561457, which is incorporated herein by reference.

“Prodrugs” and “solvates” of some components are also contemplated. The term “prodrug” means a compound (e.g., a drug precursor) that is transformed in vivo to yield the compound required by the invention, or a metabolite, pharmaceutically acceptable salt or solvate thereof. The transformation may occur by various mechanisms (e.g., by metabolic or chemical processes). A discussion of the use of prodrugs is provided by T. Higuchi and W. Stella, “Prodrugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

### Experimental Results

### Materials

Bromelain API was manufactured and provided by Mucpharm Pty Ltd (Australia) as a sterile powder. Bromelain was diluted either in phosphate buffered saline (PBS) when used as single agent, or directly in acetylcysteine solution when used as BromAc (the combination of bromelain and acetylcysteine, regardless of their respective concentrations in the combination, is referred to as “BromAc” throughout the examples), to prepare formulations of various concentrations (5, 10, 20, 25, 50, 100, 250 and 500 µg/mL). Acetylcysteine 200 mg/ml was purchased from Link Pharma (Australia) and prepared as 5, 10, 20 and 30 mg/ml solutions by dilution in PBS. The recombinant SARS-COV-2 spike protein (S1+S2 subunits) was obtained from SinoBiological (Cat#40589-V08B1). The recombinant envelope protein (Cat#MBS8309649) was obtained from MyBioSource, UK. All other reagents were of Analytical grade from Sigma Aldrich, Sydney, Australia.

### Example 1 - Gel Electrophoresis Experiments on SARS-CoV-2 Spike and Envelope Proteins

Experiments conducted by the inventors demonstrating that combinations of bromelain and NAC (Acetylcysteine) cause SARS-CoV-2 spike and envelope proteins to disintegrate will now be described. In these experiments, recombinant spike and envelope proteins were treated at a range of concentrations of single agents and BromAc (i.e. bromelain and NAC in combination), with the products being analysed by gel electrophoresis.

The spike or envelope protein was reconstituted in sterile distilled water according to the manufacturer’s instructions and aliquots were frozen at -20° C. Bromelain and Acetylcysteine stock solutions were made in in Milli-Q water. Spike or envelope protein 2.5 µg was placed in micro-centrifuge tubes and 50 µg or 100 µg/ml Bromelain, 20 mg/ml Acetylcysteine or a combination of both (i.e. BromAc) was added. The total reaction volume was 15 µL per tube. The control contained no Bromelain or Acetylcysteine.

All tubes were incubated at 37° C. for 30 min, after which 5 µl of sample buffer was added into each tube. SDS-Page precast gel from Bio-Rad was used for running the gel. Each well was loaded with 20 µL of each processed sample described above. Protein electrophoresis was performed in running buffer at 100 w for 1 hr. The gels were then immersed in Coomassie blue dye solution and gently shaken for 2 hr, after which the excess stain was removed by washing at room temperature.

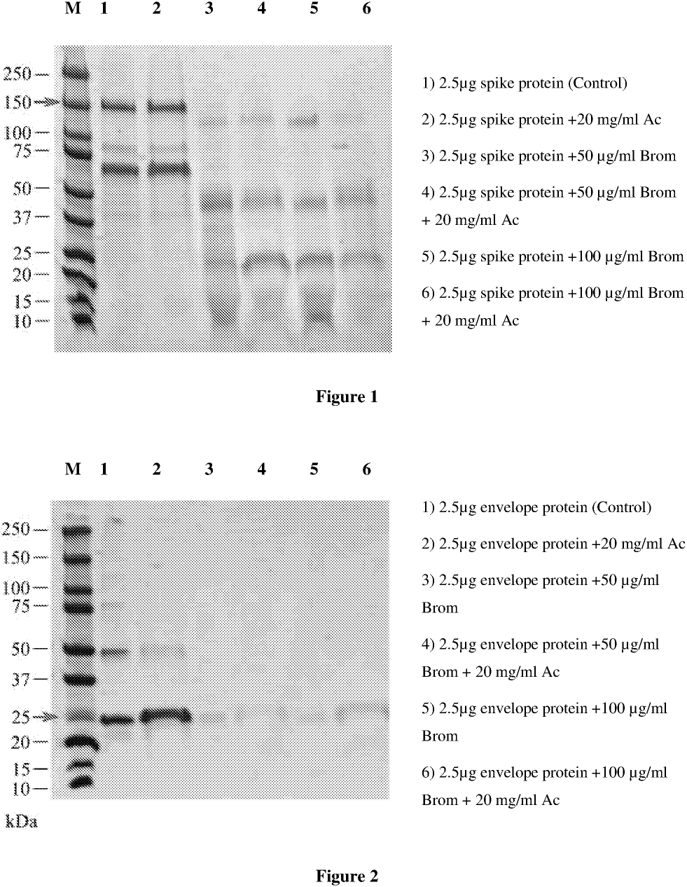

Results from the gel electrophoresis experiments indicated that with the addition of 20 mg/ml Acetylcysteine to the protein, the band showed a reduction in band thickness and intensity (FIG. 1, where the recombinant SARS-CoV-2 spike protein is marked with a red arrow), although still present, indicating that the protein has been altered but not removed (Lane 2). However, with Bromelain (50 µg/ml), the band becomes very faint (Lane 3). The combination of Bromelain (50 µg/ml) with 20 mg/ml Acetylcysteine (Lane 4) shows a very faint thin band. With the addition of 100 µg/ml Bromelain, the band is very thin and faint but still present (Lane 5). In combination, Bromelain (100 µg/ml) with 20 mg/ml Acetylcysteine (Lane 6), no visible original band but faint bands at lower molecular weight are seen, indicating fragmentation of the original protein. BromAc has affected the integrity of the spike protein by disintegration in a concentration-dependent manner. The results, however, show clear evidence of synergy between the components of BromAc.

Treatment with Acetylcysteine on the envelope protein (FIG. 2, where the recombinant SARS-CoV-2 envelope protein is marked with a red arrow) did not disintegrate the protein, however it extended sideways (Lane 1). Treatment with 50 µg/ml of Bromelain alone resulted in complete disintegration as shown by a very faint and almost absent band (Lane 3). A combination of Bromelain 50 µg/ml + Acetylcysteine 20 mg/ml also had a similar effect (Lane 4). Increasing the concentration of Bromelain to 100 µg/ml alone or in combination with Acetylcysteine 20 mg/ml also resulted in disintegration of the protein. The gel electrophoresis indicates that BromAc is effective in disintegrating the envelope protein of SARS-CoV-2 although evidence of synergy with Acetylcysteine at the concentrations tested was not observed.

The results of these experiments clearly demonstrate a synergy between bromelain and NAC in the disintegration of SARS-CoV-2 spike proteins, and that envelope proteins are disintegrated by bromelain. The inventors believe that these data support the proposal that administering a therapeutically effective amount of a combination of a glycoprotein affecting protease (e.g. bromelain) and a disulphide bond breaking agent (e.g. NAC) may be effective for the prophylaxis or treatment of a viral infections.

### Example 2 -The Effect of Acetylcysteine on the Disulphide Bonds in Spike Protein

Recombinant SARS-CoV-2 spike protein at a concentration of 3.0 µg/ml in phosphate buffer saline (PBS) (pH7.0) containing 1 mM (EDTA) was prepared. A series of similar tubes were prepared (7 × 2). To one set of tubes 0, 10, 20, 40 & 50 µl of Acetylcysteine was added and agitated at 37° C. for 30 min, followed by equivalent addition of DTT (Dithiotretiol) (0.5 M) and further agitated for 30 min at 37° C. To the next (control) set of tubes containing spike protein, only DTT (0.5 M) was added as before, without any Acetylcysteine, and the tubes then agitated at 37° C. for 30 min. The absorbance was then read at 310 nm.

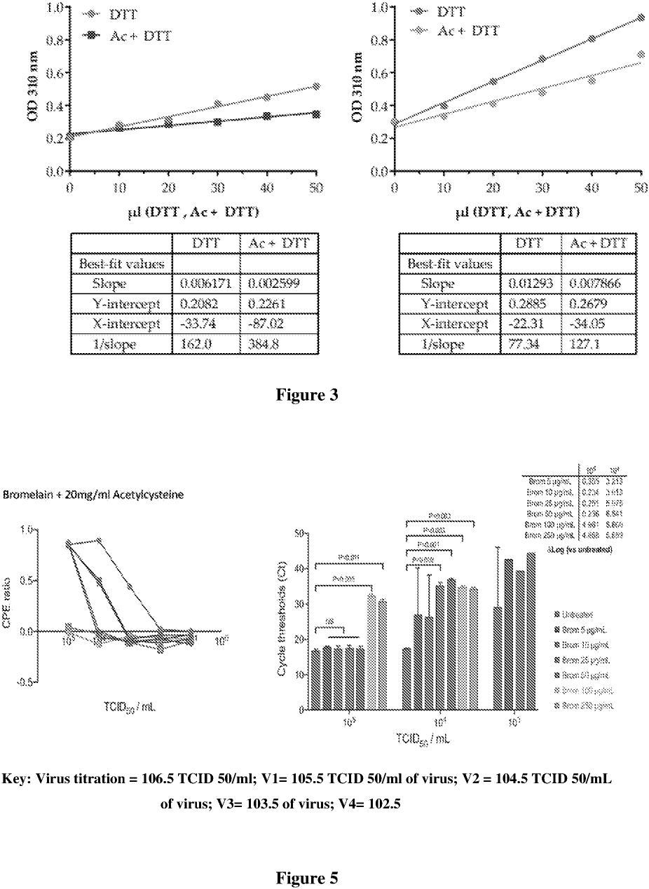

The comparative reduction of disulphide bonds on the spike protein between DTT alone and DTT with Acetylcysteine demonstrated a 42% difference (FIG. 3B), based on the slope of the graphs [0.002599/0.006171 (100) = 42 %]. Acetylcysteine was thus able to reduce 58% of the disulphide linkages in the sample, after which the remaining disulphide bonds were reduced by DTT to produce the chromogen that was monitored in the spectra. Similarly, the differential assay between Acetylcysteine and DTT for the reduction of disulphide bonds found in the envelope protein [0.007866/0.01293 (100) = 60%] indicates that Acetylcysteine reduces 40% of the disulphide bonds before the addition of DTT (FIG. 3C).

This assay indicates that the disulphide bonds were lysed by Acetylcysteine, and hence potential targets for BromAc for treating (disinfecting) the SARS-CoV2 virus.

### Example 3 - Protective Effect of BromAc on Vero and BGM Cells

Live SARS-CoV-2 virus (SARS-COV-2 R209112 strain) was pre-treated with BromAc, Bromelain or Acetylcysteine, at a range of concentrations, prior to adding to Vero and BGM cells for infection. Cell microscopy, staining and qRT-PCR were performed to examine the effects of the virus on the cells.

SARS-COV-2 R209112 strain were cultured at 1 MOI to 10⁻⁴. The SARS-CoV-2 inactivation tests were conducted with various concentrations of Bromelain alone (0, 5, 10, 25, 50, 100 and 500 µg/mL), Acetylcysteine alone (0, 5, 10, 20 mg/ml) and BromAc combinations (all including 20 mg/ml Acetylcysteine) with 10-fold serial TCID50/mL dilutions of the virus.

Following 1 hour of drug exposure to the virus at 37° C., inoculation of all samples in duplicate on confluent Vero and GBM cells (ATCC) was performed and the samples incubated for 5 days at 36° C. with 5% CO₂. Cells were maintained in Eagle’s minimal essential medium (EMEM) with 2% Penicillin-Streptomycin, 1% L-glutamine, and 2% inactivated foetal bovine serum. Results were obtained by: (1) daily optical microscopy observations, (2) quantitative reverse-transcriptase polymerase chain reaction (qRT-PCR) of supernatant extracts, and (3) an end-point cytotoxicity assay.

Briefly, the RNA from each of the sample’s supernatants was extracted by the semi-automated eMAG® workstation (bioMérieux, Lyon, FR), and RdRp IP2-targeted RdRp Institute Pasteur qRT-PCR was performed on a QuantStudio™ 5 System (Applied Biosystems, Thermo Fisher Scientific). The ΔLOG of viral replication was calculated by the difference between treated and untreated wells per condition (1 log ≈ 3 PCR Ct). The end-point cytotoxicity assay consisted of adding neutral red dye (Merck KGaA, Darmstadt, DE) to the cell monolayers, incubating at 37° C. for 45 minutes, washing with PBS, and adding citrate ethanol before optical density (OD) was measured at 540 nm (Labsystems Multiskan Ascent Reader, Thermo Fisher Scientific). A cytopathic effect (CPE) ratio was calculated by taking the complement of the average of treated cells divided by the average of untreated cells.

### Vero

In the Vero cells, the cytopathic effect at 5 days of various concentrations of Bromelain with Acetylcysteine at a standardised concentration of 20 mg/ml (BromAc) indicated that between Bromelain 5-50 µg/ml, there was no therapeutic effect with MOI 1. The effect began to show with MOI⁻¹ at concentrations of 25-100 µg/ml Bromelain. With the addition of 250 µg/ml Bromelain, a protective effect is seen from MOI 1 onwards. There was no indication of cytotoxic effect on the host cells of BromAc at all concentrations investigated. The addition of Acetylcysteine alone did not show any therapeutic effect at 20 mg/ml at MOI 1, MOI⁻¹ and Mol⁻². Optical results are shown in Table 1A.

TABLE 1A

Vero optical microscope observation of SARS-CoV-2 following BromAc treatment at various concentrations of Bromelain and Acetylcysteine at 20 mg/ml (in duplicate)

| | | | | | | | | | | | | |
|---|---|---|---|---|---|---|---|---|---|---|---|---|
| VIRUS | V1 | | V2 | | V3 | | V4 | | V5 | | TOXICITY | |
| Virus untreated control | + | + | + | + | + | + | - | - | - | - | Nil | Nil |
| Control | | | | | | | | | | | | |
| 5 µg/ml Brom + 20 mg/ml Ac | + | + | + | + | - | - | - | - | - | - | Nil | Nil |
| 10 µg/ml Brom + 20 mg/ml Ac | + | + | + | - | - | - | - | - | - | - | Nil | Nil |
| 25 µg/ml Brom + 20 mg/ml Ac | + | + | - | - | - | - | - | - | - | - | Nil | Nil |
| 50 µg/ml Brom + 20 mg/ml Ac | + | + | - | - | - | - | - | - | - | - | Nil | Nil |
| 100 µg /ml Brom + 20 mg/ml Ac | - | - | - | - | - | - | - | - | - | - | Nil | Nil |
| 250 µg/ml Brom + 20 mg/ml Ac | - | - | - | - | - | - | - | - | - | - | Nil | Nil |
| 20 mg/ml Ac | + | + | + | + | - | + | - | - | - | - | Nil | Nil |
| 100 µg/ml Brom | + | + | + | + | - | - | - | - | - | - | Nil | Nil |

Key: ‘+’ indicates SARS-CoV-2 infection, ‘-’indicates no infection, V1 = MOI 1, V2 = MOI⁻¹, etc. in duplicate, Brom = Bromelain, Ac = Acetylcysteine, shading = cytopathic effect of virus on cell

The results from the neutral red staining indicate similar results (as shown in Table 1B). The control row indicates normal growth of Vero cells that are uninfected. Shading indicates cytopathic effect from the SARS-CoV-2 virus.

TABLE 1B

Results of cytopathic effect by neutral red staining of Vero cells following BromAc treatment at various concentrations of Bromelain and Acetylcysteine at 20 mg/ml (in duplicate)

| VIRUS | V1 | | V2 | | V3 | | V4 | | V5 | | TOXICITY | |
|---|---|---|---|---|---|---|---|---|---|---|---|---|
| Virus untreated control | 0.197 | 0.382 | 0.216 | 0.207 | 2.013 | 0.205 | 1.982 | 1.906 | 2.003 | 1.959 | 2.031 | 1.729 |
| Control | 1.29 | 2.012 | 2.027 | 2.027 | 2.161 | 2.216 | 2.095 | 2.095 | 2.052 | 2.065 | 2.118 | 1.847 |
| 5 µg/ml Brom + 20 mg/ml Ac | 0.242 | 0.362 | 1.838 | 0.130 | 2.156 | 2.102 | 2.136 | 1.971 | 1.986 | 2.131 | 2.114 | 1.683 |
| 10 µg/ml Brom + 20 mg/ml Ac | 0.291 | 0.269 | 0.173 | 1.907 | 2.076 | 2.241 | 2.349 | 2.063 | 2.056 | 2.202 | 2.269 | 1.871 |
| 25 µg/ml Brom + 20 mg/ml Ac | 0.265 | 0.303 | 2.257 | 1.934 | 2.156 | 2.189 | 2.22 | 2.051 | 2.154 | 1.928 | 2.143 | 1.771 |
| 50 µg/ml Brom + 20 mg/ml Ac | 0.261 | 0.239 | 2.027 | 1.926 | 2.200 | 2.217 | 2.178 | 2.072 | 2.193 | 2.282 | 2.244 | 1.878 |
| 100 µg/ml Brom + 20 mg/ml Ac | 1.849 | 2.109 | 2.079 | 2.326 | 1.990 | 2.109 | 2.102 | 2.054 | 2.170 | 2.041 | 2.078 | 2.155 |
| 250 µg/ml Brom + 20 mg/ml Ac | 1.667 | 2.137 | 1.978 | 2.081 | 2.239 | 2.162 | 2.28 | 2.398 | 2.117 | 2.209 | 2.208 | 1.925 |
| 20 mg/ml Ac | 0.256 | 0.316 | 0.278 | 0.201 | 2.035 | 0.440 | 1.936 | 1.93 | 1.986 | 1.902 | 1.911 | 1.616 |
| 100 µg/ml Brom | 0.127 | 0.180 | 0.507 | 1.155 | 1.814 | 1.968 | 2.162 | 2.150 | 2.110 | 2.060 | 2.006 | 2.343 |

Key: V1 = MOI 1, V2 = MOI⁻¹, etc. in duplicate, Brom = Bromelain, Ac = Acetylcysteine, blue shading = cytopathic effect of virus on cell, shading = normal growth of control cells

The CPE ratio in Table 1C indicates there is minimal effect of BromAc at MOI 1 at all concentrations except 250 µg/ml Bromelain. With MOI⁻¹, there is slight effect seen with Bromelain between 5 and 10 µg/ml, however, the effect is complete with BromAc at a range of Bromelain 25-250 µg/ml. At MOI⁻², a therapeutic effect is seen at all concentrations of BromAc.

TABLE 1C

- CPE Ratio of BromAc in Vero at varying concentrations of Bromelain combined with Acetylcysteine 20 mg/ml and Acetylcysteine as a single agent (20 mg/ml)

| CPE RATIO | V1 (MOI 1) | V2 (MOI⁻¹) | V3 (MOI ⁻²) | V4 (MOI⁻³) | V5 (MOI⁻⁴) |
|---|---|---|---|---|---|
| Virus untreated control | 0.854 | 0.893 | 0.441 | 0.020 | 0.001 |
| 5 µg/ml Brom + 20 mg/ml Ac | 0.848 | 0.504 | -0.074 | -0.035 | -0.038 |
| 10 µg/ml Brom + 20 mg/ml Ac | 0.859 | 0.476 | -0.088 | -0.112 | -0.074 |
| 25 µg/ml Brom + 20 mg/ml Ac | 0.857 | -0.057 | -0.095 | -0.077 | -0.029 |
| 50 µg/ml Brom + 20 mg/ml Ac | 0.874 | -0.001 | -0.114 | -0.071 | -0.128 |
| 100 µg/ml Brom + 20 mg/ml Ac | -0.009 | -0.123 | -0.045 | -0.060 | -0.074 |
| 250 µg/ml Brom + 20 mg/ml Ac | 0.041 | -0.023 | -0.110 | -0.179 | -0.091 |
| 20 mg/mL Ac | 0.856 | 0.879 | 0.376 | 0.117 | 0.132 |
| 100 µg/ml Brom | 0.931 | 0.625 | 0.148 | 0.028 | 0.060 |

Key: V1 = MOI 1, V2 = MOI ⁻¹, etc., Brom = Bromelain, Ac = Acetylcysteine, shading indicates therapeutic effect of drug treatment

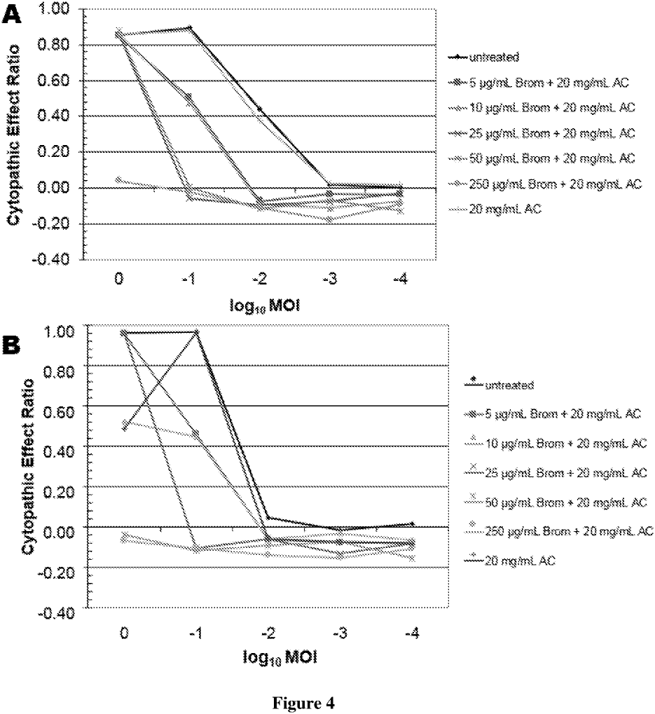

The cytopathic effect of BromAc with Bromelain at varying concentrations and just Acetylcysteine (20 mg/ml), compared to untreated Vero cells was examined when added to MOI ¹ to MOI⁻² (FIG. 4A, x-axis as 1.2). Noticeably, the addition of 100 to 250 µg/ml BromAc did not allow the virus to replicate from MOI 1 with 25 to 50 µg/ml showing the same effect from MOI⁻¹. FIG. 5 indicates the statistical differences between the concentrations by cycle threshold (Ct).

In FIG. 5, the impact of bromelain and acetylcysteine treatment on SARS-CoV-2 cytopathic effect and level of replication when cultured in-vitro at different dilutions in Vero cells is shown. The results are expressed as Cycle threshold (Ct) needed to obtain significant viral RNA detected are shown on the bar graph on the right. Bar charts show the mean (SD). Statistical significances compared with control using two-way ANOVA followed by a procedure of Benj amini, Krieger and Yekutieli multiple-comparison test (Vero cells infected with untreated SARS-CoV-2) are shown with exact p values.

### BGM

The cytopathic effect at 7 days of various concentrations of Bromelain with a standardised concentration of Acetylcysteine 20 mg/ml (BromAc) was also examined on BGM cells. The results indicate that between 5-25 µg/ml Bromelain, there was no therapeutic effect at MOI 1. An effect was evident from MOI⁻¹ with increasing concentrations from Bromelain 25 µg/ml. With the addition of 50, 100 and 250 µg/ml Bromelain, an effect is seen from MOI 1. There was no indication of a cytotoxic effect to the cells of BromAc at any of the concentrations investigated. The addition of only Acetylcysteine (20 mg/ml) did not show any effect alone at MOI 1 and MOI⁻¹. Optical results are shown in Table 2A.

TABLE 2A

- BGM optical microscope observation of SARS-Co V-2 following BromAc treatment at various concentrations of Bromelain and Acetylcysteine at 20 mg/ml (in duplicate)

| | | | | | | | | | | | | |
|---|---|---|---|---|---|---|---|---|---|---|---|---|
| VIRUS | V1 | | V2 | | V3 | | V4 | | V5 | | TOXICITY | |
| Virus untreated control | + | + | + | + | - | - | - | - | - | - | Nil | Nil |
| Control | | | | | | | | | | | | |
| 5 µg/ml Brom + 20 mg/ml Ac | + | + | + | + | - | - | - | - | - | - | Nil | Nil |
| 10 µg/ml Brom + 20 mg/ml Ac | | + | + | - | - | - | - | - | - | - | Nil | Nil |
| 25 µg/ml Brom + 20 mg/ml Ac | + | + | - | - | - | - | - | - | - | - | Nil | Nil |
| 50 µg/ml Brom + 20 mg/ml Ac | - | - | - | - | - | - | - | - | - | - | Nil | Nil |
| 100 µg /ml Brom + 20 mg/ml Ac | - | - | - | - | - | - | - | - | - | - | Nil | Nil |
| 250 µg/ml Brom + 20 mg/ml Ac | - | - | - | - | - | - | - | - | - | - | Nil | Nil |
| 20 mg/ml Ac | + | + | + | + | - | - | - | - | - | - | Nil | Nil |
| 100 µg/ml Brom | + | + | + | + | + | + | + | + | + | + | Nil | Nil |

Key: ‘+’ indicates SARS-CoV-2 infection, ‘-’indicates no infection, V1 = MOI 1, V2 = MOI⁻¹, etc. in duplicate, Brom = Bromelain, Ac = Acetylcysteine, shading = cytopathic effect of virus on cell

The results from the neutral red staining indicate similar results, which are shown in Table 2B and confirm these findings. The control row indicates normal growth of BGM cells that are uninfected. Shading indicates cytopathic effect from the SARS-CoV-2 virus. No cytotoxicity was shown by BromAc at all concentrations investigated.

TABLE 2B

Results of cytopathic effect by neutral red staining of BGM cells following BromAc treatment at various concentrations of Bromelain and Acetylcysteine at 20 mg/ml (in duplicate)

| VIRUS | V1 | | V2 | | V3 | | V4 | | V5 | | TOXICITY | |
|---|---|---|---|---|---|---|---|---|---|---|---|---|
| Virus untreated control | 0.099 | 0.081 | 0.081 | 0.071 | 2.341 | 2.27 | 2.497 | 2.413 | 2.391 | 2.356 | 2.276 | 1.404 |
| Control | 2.16 | 2.557 | 2.59 | 2.659 | 2.54 | 2.587 | 2.653 | 2.466 | 2.383 | 2.6 | 2.751 | 2.172 |
| 5 µg/ml Brom + 20 mg/ml Ac | 0.113 | 0.089 | 0.108 | 2.493 | 2.43 | 2.682 | 2.722 | 2.482 | 2.544 | 2.665 | 2.952 | 2.008 |
| 10 µg/ml Brom + 20 mg/ml Ac | 2.211 | 0.094 | 2.593 | 0.083 | 2.487 | 2.628 | 2.643 | 2.335 | 2.544 | 2.599 | 2.646 | 2.341 |
| 25 µg/ml Brom + 20 mg/ml Ac | 0.093 | 0.101 | 2.671 | 2.661 | 2.624 | 2.501 | 2.635 | 2.82 | 2.57 | 2.654 | 2.665 | 2.39 |
| 50 µg/ml Brom + 20 mg/ml Ac | 2.261 | 2.759 | 2.792 | 2.599 | 2.634 | 2.643 | 2.636 | 2.554 | 2.745 | 2.825 | 2.938 | 2.186 |
| 100 µg/ml Brom + 20 mg/ml Ac | 2.099 | 2.728 | 2.565 | 2.756 | 2.716 | 2.951 | 2.515 | 2.675 | 2.908 | 2.678 | 2.880 | 2.529 |
| 250 µg/ml Brom + 20 mg/mL Ac | 2.285 | 2.88 | 2.714 | 2.624 | 2.857 | 2.649 | 2.749 | 2.817 | 2.599 | 2.753 | 2.752 | 2.451 |
| 20 mg/ml Ac | 0.099 | 0.112 | 0.099 | 0.082 | 1.409 | 2.528 | 2.62 | 2.571 | 2.605 | 2.628 | 2.486 | 1.424 |
| 100 µg/ml Brom | 0.082 | 0.094 | 0.086 | 0.094 | 1.020 | 0.125 | 2.439 | 2.502 | 2.411 | 2.466 | 2.602 | 2.672 |

Key: V1 = MOI 1, V2 = MOI⁻¹, etc. in duplicate, Brom = Bromelain, Ac = Acetylcysteine, blue shading = cytopathic effect of virus on cell, shading = normal growth of control cells

The CPE ratio in FIG. 4B and Table 2C indicates there is minimal effect of BromAc at MOI 1 at concentrations lower than 50 µg/ml Bromelain. With MOI⁻¹, there is slight effect seen with Bromelain between 5 and 10 µg/ml, however, the effect is complete with BromAc at a range of Bromelain 25-250 µg/ml. At MOI⁻², a therapeutic effect is seen at all concentrations of BromAc.

TABLE 2C

CPE Ratio of BromAc in BGM at varying concentrations of Bromelain combined with Acetylcysteine 20 mg/ml and Acetylcysteine as a single agent (20 mg/ml)

| CPE RATIO | V1 (MOI 1) | V2 (MOI⁻¹) | V2 (MOI ⁻²) | V3 (MOI⁻³) | V4 (MOI⁻⁴) |
|---|---|---|---|---|---|
| Virus untreated control | 0.963 | 0.969 | 0.045 | -0.017 | 0.017 |
| 5 µg/ml Brom + 20 mg/ml Ac | 0.958 | 0.461 | -0.059 | -0.078 | -0.079 |
| 10 µg/ml Brom + 20 mg/ml Ac | 0.523 | 0.446 | -0.059 | -0.031 | -0.065 |
| 25 µg/ml Brom + 20 mg/ml Ac | 0.960 | -0.104 | -0.061 | -0.130 | -0.082 |
| 50 µg/ml Brom + 20 mg/ml Ac | -0.040 | -0.117 | -0.093 | -0.075 | -0.154 |
| 100 µg/ml Brom + 20 mg/ml Ac | 0.064 | -0.032 | -0.099 | -0.006 | -0.083 |
| 250 µg/ml Brom + 20 mg/ml Ac | -0.070 | -0.106 | -0.140 | -0.153 | -0.108 |
| 20 mg/ml Ac | 0.960 | 0.835 | 0.460 | -0.024 | -0.089 |
| 100 µg/ml Brom | 0.967 | 0.965 | 0.783 | 0.064 | 0.076 |

Key: V1 = MOI 1, V2 = MOI ⁻¹, etc., Brom = Bromelain, Ac = Acetylcysteine, shading indicates therapeutic effect of drug treatment

The cytopathic effect of BromAc with Bromelain at varying concentrations in combination with Acetylcysteine (20 mg/ml) or just Acetylcysteine (20 mg/ml), compared to untreated BGM cells at 7 days post treatment was examined when added to MOI 1 to MOI^{-2.} The addition of 50 and 250 µg/ml BromAc did not allow the virus to replicate from MOI 1 with 25 to 50 µg/ml showing the same effect from MOI⁻¹.

Based on the guidelines of viral inactivation established by the World Health Organization, a robust and reliable process of inactivation will be able to inactivate 4 logs or more [(Δlog = Ct treated - Ct untreated)/3; as 1 log≈3 Ct]. At MOI 1, after treatment with BromAc, Δlog averaged 3.969 and 3.058 on BGM and Vero cells, respectively. At MOI⁻¹ and MOI⁻² on BGM cells, the Δlog averaged 5.734 and 6.86 for all BromAc concentrations, respectively. At MOI 1 and MOI⁻¹ on Vero cells, the Δlog was 4.961 and 5.660 for 100 µg/ml BromAc (Table 3).

TABLE 3

SARS-CoV-2 x BromAc viral inactivation test (RNA extraction) in Vero cells

| | V1 | V2 |
|---|---|---|
| Virus untreated control | 0.037 | 0.015 |
| 5 µg/ml Brom + 20 mg/ml Ac | 0.355 | 3.213 |
| 10 µg/ml Brom + 20 mg/ml Ac | 0.234 | 3.013 |
| 25 µg/ml Brom + 20 mg/ml Ac | 0.251 | 5.975 |
| 50 µg/ml Brom + 20 mg/ml Ac | 0.236 | 6.541 |
| 100 µg/ml Brom + 20 mg/ml Ac | 4.961 | 5.660 |
| 250 µg/ml Brom + 20 mg/ml Ac | 4.668 | 5.137 |
| 20 mg/mL Ac | 0.088 | 0.224 |
| 100 µg/ml Brom | 0.072 | 0.345 |

Key: V1 = MOI 1, V2 = MOI ⁻¹, etc. in duplicate, Brom = Bromelain, Ac = Acetylcysteine; shading indicates a difference of at least 4 logs vs control indicating viral inactivation

Further confirmation of BromAc efficacy was observed in photographs taken of slides where Vero cells exposed to the live SARS-CoV-2 virus were either not treated (control) or treated with BromAc at a low concentration of 50 µg Bromelain/20 mg/ml Acetylcysteine. The results showed that in the control, the virus was cytotoxic to the host cells. However, when treated with BromAc there was no infection of the cells and also no cytotoxic effect. These studies using qRT-PCR and staining techniques confirms the anti-viral action of BromAc that may be translated to clinical application.

### Summary of Experimental Findings (Examples 1 to 3)

The inventors’ first study on the spike protein using gel-electrophoresis showed that these proteins were hydrolysed into fragments. Subsequent studies using UV spectroscopy to investigate the reductive action of NAC indicated that it reduces the disulfide bonds found within cysteine residues in the spike protein. The results indicated that BromAc can affect the molecular geometry of the spike protein that contains essential domains S1 and S2, which are vital for fusion after binding to the ACE2 receptors. Further investigation on the envelope protein indicated a similar result, that BromAc also disintegrates the protein.

With these preliminary results, the inventors commissioned the in vitro evaluation on live SARS-CoV-2 virus infectivity in Vero and BGM cells. In summary, in these live virus tests, pretreatment of SARS-CoV-2 with BromAc at various low concentrations prevented infection in Vero and BGM cells. A concentration dependent response was seen.

The live virus studies showed that Bromelain (50-500 µg/ml plus 20 mg/ml Acetylcysteine, i.e. BromAc) completely prevented infection at MOI⁻¹ to MOI^{-2.} This assessment was based on the -log delta values generated that were above 4.0 (stipulation by WHO as effective antiviral agents). At MOI 1, the values were close to 4.0 (3.6), again indicating that even at very high viral infection, BromAc was effective. Cytopathic effects were observed for SARS-CoV-2 virus controls at MOI 1, MOI⁻¹, and MOI⁻² on both BGM and Vero cells. The BromAc combination in vitro showed inactivation of the virus by preventing the cytopathic effect on two cell lines and yielding no viral RNA replication. These results suggest to the inventors that BromAc could be evaluated as an early treatment of SARS-CoV-2 infection, potentially able to prevent the progression towards severe forms of the disease and reduce the risk of infection to patient contacts.

The inventors believe that as BromAc disintegrates the spike from SARS-CoV-2 and renders it non-infective in Vero and BGM cells, nasal administration may be therapeutic in patients with early SARS-CoV-2 infection. Investigations are continuing.

### Example 4A - SARS-CoV-2 Whole Virus Inactivation With BromAc

Fully respecting the World Health Organization (WHO) interim biosafety guidance related to the coronavirus disease, the SARS-CoV-2 whole virus inactivation tests were carried out with a wild-type (WT) strain representative of early circulating European viruses (GISAID accession number EPI_ISL_578176). A second SARS-CoV-2 strain (denoted as ΔS), reported through routine genomic surveillance in the Auvergne-Rhône-Alpes region of France, was added to the inactivation tests due to a rare mutation in the spike S1/S2 cleavage site and its culture availability in the laboratory (GISAID accession number EPI_ISL_578177).

These tests were conducted with incremental concentrations of Bromelain alone (0, 25, 50, 100, and 250 µg/mL), Acetylcysteine alone (20 mg/mL), and with formulations including different Bromelain concentrations combined with a constant 20 mg/mL Acetylcysteine (i.e. BromAc), against two virus culture dilutions at 10^{5.5} and 10^{4.5} TCID₅₀/mL. Following 1 h of drug exposure at 37° C., all conditions, including the control, were diluted 100-fold to avoid cytotoxicity, inoculated in quadruplicate on confluent Vero cells (CCL-81; ATCC©, Manassas, VA, USA), and incubated for 5 days at 36° C. with 5% CO₂. Cells were maintained in Eagle’s minimal essential medium (EMEM) with 2% Penicillin-Streptomycin, 1% L-glutamine, and 2% inactivated fetal bovine serum. Results were obtained by daily optical microscopy observations, an end-point cell lysis staining assay, and reverse-transcriptase polymerase chain reaction (RT-PCR) of supernatant RNA extracts. Briefly, the end-point cell lysis staining assay consisted of adding Neutral Red dye (Merck KGaA, Darmstadt, Germany) to cell monolayers, incubating at 37° C. for 45 min, washing with PBS, and adding citrate ethanol before optical density (OD) was measured at 540 nm (Labsystems Multiskan Ascent Reader, Thermo Fisher Scientific, Waltham, MA, USA). OD was directly proportional to viable cells, so a low OD would signify important cell lysis due to virus replication. In addition, RNA from well supernatants was extracted by the semi-automated eMAG® workstation (bioMérieux, Lyon, FR), and SARS-CoV-2 RdRp IP2-targeted RdRp Institute Pasteur RT-PCR was performed on a QuantStudio™ 5 System (Applied Biosystems, Thermo Fisher Scientific, Foster City, CA, USA). Log₁₀ reduction values (LRV) of viral replication were calculated by the difference between treatment and control wells per condition divided by 3.3 (as 1 log10, approx. 3.3 PCR Cycle thresholds (Ct)).

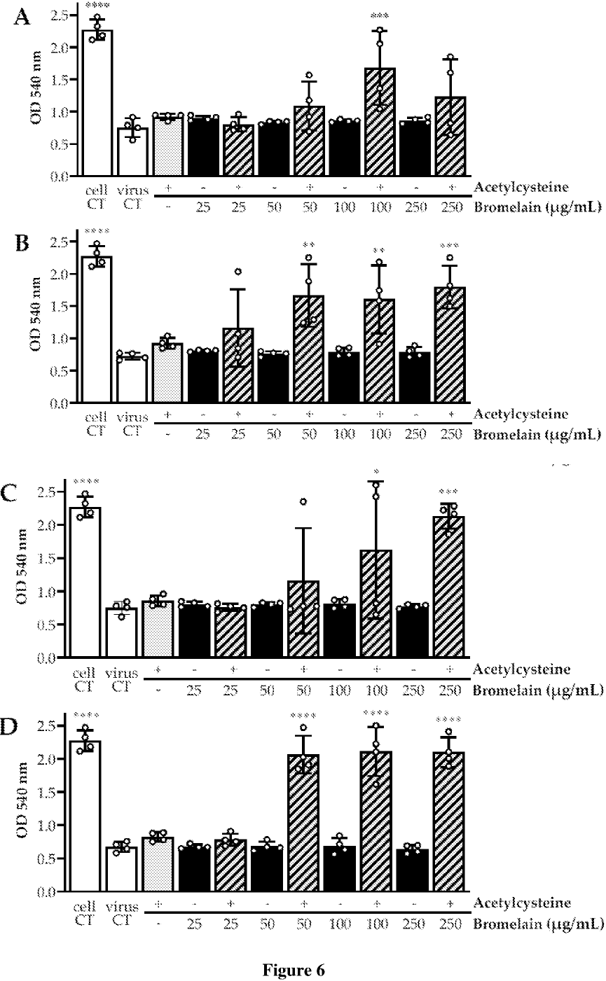

For both SARS-CoV-2 strains tested, the untreated virus controls at 10^{5.5} and 10^{4.5} TCID₅₀/mL yielded typical cytopathic effects (CPE), and no cytotoxicity was observed for any of the drug combinations on Vero cells. Optical CPE results were invariably confirmed by end-point Neutral Red cell staining. Overall, Bromelain and Acetylcysteine treatment alone showed no viral inhibition, all with CPE comparable to virus control wells, whereas BromAc combinations displayed virus inactivation in a concentration dependent manner (FIG. 6). Treatment on 10^{4.5} TCID₅₀/mL virus titers (FIGS. 6B,D) yielded more consistent inhibition of CPE for quadruplicates than on 10^{5.5} TCID₅₀/mL virus titers (FIGS. 6A,C).

As shown in FIG. 6, cell lysis assays demonstrated in vitro inactivation potential of Acetylcysteine and Bromelain combined (BromAc) against SARS-CoV-2. Cell viability was measured by cell staining with Neutral Red, where optical density (OD) is directly proportional to viable cells. Low OD would signify important cell lysis due to virus replication. The wild-type (WT) SARS-CoV-2 strain at 5.5 and 4.5 log₁₀TCID₅₀/mL titers (FIGS. 6A and B, respectively) showed no inhibition of cytopathic effect (CPE) for single agent treatment, compared to the mock treatment virus control condition. BromAc combinations were able to inhibit CPE, compared to the mock infection cell controls. Treatment of a SARS-CoV-2 spike protein variant (ΔS) with a mutation at the S1/S2 junction at 5.5 and 4.5 log10TCID50/mL titers (FIGS. 6C and D, respectively) showed similar results. Bars represent the average of each quadruplicate per condition, illustrated by white circles. Ordinary one-way ANOVA was performed, using the mock treatment virus control as the control condition (****p < 0.0001, ***p < 0.0005, **p < 0.003, and *p < 0.05).

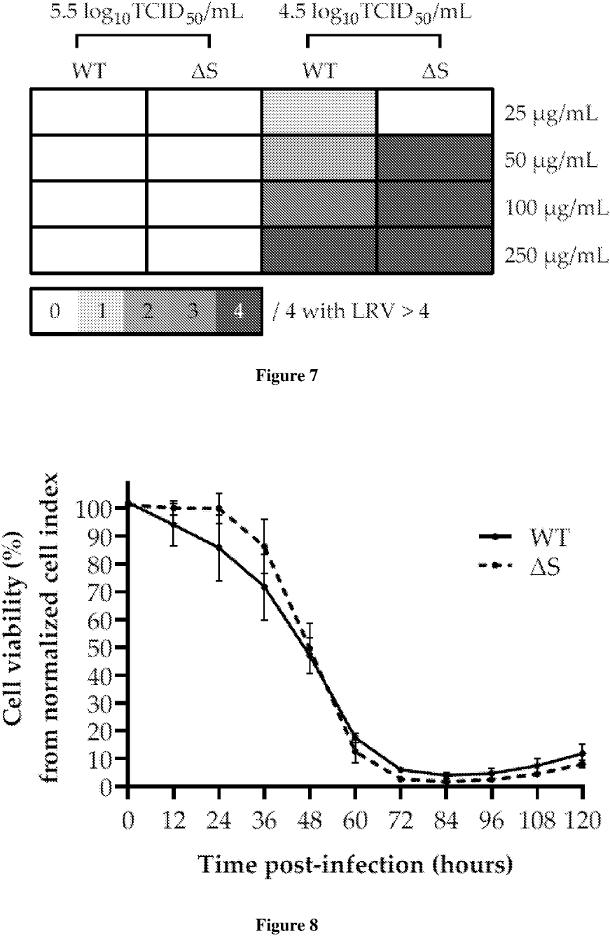

Based on the virus inactivation guidelines established by the WHO, a robust and reliable process of inactivation will be able to reduce replication by at least 4 logs [Log₁₀ reduction value (LRV) = (RT-PCR Ct treatment - RT-PCR Ct control)/3.3; as 1 log₁₀ is approx. 3.3 Ct]. As such, RT-PCR was performed on the RNA extracts to directly measure virus replication. For the wild-type (WT) strain at 10^{4.5} TCID₅₀/mL, successful LRV > 4 were observed with 1 out of 4 wells, 2 out of 4 wells, 3 out of 4 wells, and 4 out of 4 wells for 25, 50, 100 and 250 µg/20 mg/mL BromAc, respectively (FIG. 7). It is worth noting that at 10^{5.5} TCID₅₀/mL, LRV were slightly below the threshold at, on average, 3.3, with 3 out of 4 wells and 2 out of 4 wells for 100 and 250 µg/20 mg/mL BromAc, respectively (Table 4, see below). For the spike protein mutant (ΔS) at 10^{4.5} TCID₅₀/mL, no successful LRV > 4 was observed for 25 µg/20 mg/mL BromAc, but it was observed in 4 out of 4 wells for 50, 100, and 250 µg/20 mg/mL BromAc (FIG. 7). Of note, at 10^{5.5} TCID₅₀/mL, LRV were slightly below the threshold at, on average, 3.2, with 1 out of 4 wells, 2 out of 4 wells, and 4 out of 4 wells for 50, 100, and 250 µg/20 mg/mL BromAc, respectively (Table 4). Overall, in vitro inactivation of both SARS-CoV-2 strains’ replication capacity was observed in a dose-dependent manner, most strongly demonstrated at 100 and 250 µg/20 mg/mL BromAc against 10^{4.5} TCID₅₀/mL of virus.

FIG. 7 shows the threshold matrix of log10 reduction values (LRV) of in vitro virus replication 96 h after BromAc treatment on WT and ΔS SARS-CoV-2 strains at 5.5 and 4.5 log₁₀TCID₅₀/mL titers. LRV were calculated with the following formula: LRV = (RT-PCR Ct of treatment—RT-PCR Ct virus control)/3.3; as 1 logic₁₀ is approx. 3.3 Ct. The color gradient matrix displays the number of quadruplicates per condition yielding an LRV > 4, corresponding to a robust inactivation according to the WHO. In the table, WT = wild-type; ΔS = S1/S2 spike mutant.

Table 4, set out below, shows log₁₀ reduction values (LRV) of in vitro virus replication 96 h after BromAc treatment on WT and ΔS SARS-CoV-2 strains at 5.5 and 4.5 log₁₀TCID₅₀/mL titers. LRV were calculated with the following formula: LRV = (RT-PCR Ct of treatment - RT-PCR Ct virus control)/3.3; as 1 log₁₀ is approx.. 3.3 Ct. Each replicate is described. TCID₅₀/mL = Median Tissue Culture Infectious Dose; WT = wild-type; ΔS = S1/S2 spike mutant.

TABLE 4

| BromAc (µg/20 mg/mL) | | Virus Titer | |
|---|---|---|---|
| | | 5.5 log₁₀TCID₅₀/mL | 4.5 log₁₀TCID₅₀/mL |
| WT | 25 | 0.033 0.104 0.250 0.213 | 0.463 0.356 4.390 0.173 |
| | 50 | 0.050 0.304 0.446 0.698 | 0.471 4.378 0.404 4.651 |
| | 100 | 3.415 3.323 0.360 3.313 | 4.418 4.463 0.423 4.508 |
| | 250 | 0.033 3.423 0.200 3.389 | 4.496 4.370 4.419 4.506 |
| ΔS | 25 | 0.010 0.153 NA 0.414 | 0.330 0.313 0.172 0.075 |
| | 50 | 3.252 0.297 0.278 0.275 | 4.7624.612 4.61.8 4.571 |
| | 100 | 3.191 3.260 0.210 0.301 | 6.054 4.518 5.155 4.747 |
| | 250 | 3.287 3.298 3.308 3.308 | 4.333 4.302 4.410 4.361 |

### Example 4B - Replication Kinetics by Real-Time Cell Analysis

To compare the in vitro replication capacity of both WT and ΔS SARS-CoV-2 strains, replication kinetics were determined by measuring the electrode impedance of microelectronic cell sensors on the xCELLigence Real-Time Cell Analyzer (RTCA) DP Instrument (ACEA Biosciences, Inc., San Diego, CA, USA). Vero cells were seeded at 20,000 cells per well on an E-Plate 16 (ACEA Biosciences, Inc., San Diego, CA, USA) and incubated with the same media conditions as described previously at 36° C. with 5% CO2. After 24 h, SARS-CoV-2 culture isolates were inoculated in triplicate at a multiplicity of infection of 10-2. Mock infections were performed in quadruplicate. Electronic impedance data (cell index) were continuously collected at 15-minute intervals for 6 days. Area under the curve analysis of normalized cell index, established at time of inoculation, was then calculated at 12-hour intervals. At each interval, cell viability was determined by normalizing against the corresponding cell control. Tukey multiple comparison tests were used to compare each condition on GraphPad Prism (software version 9.0; San Diego, CA, USA).

SARS-CoV-2 replication capacity of WT and ΔS SARS-CoV-2 were measured by Real-Time Cell Analysis. As can be seen in FIG. 8, real-time cell analysis demonstrated comparable replication kinetics for both WT and ΔS SARS-CoV-2 strains. No significant difference in cell viability was observed between WT and ΔS at any time point. From 48 h post-infection, WT and ΔS cell viability were significantly different compared to the mock infection (p < 0.05).

In FIG. 8, data points correspond to area under the curve analysis of normalized cell index (electronic impedance of RTCA established at time of inoculation) at 12-hour intervals. Cell viability was then determined by normalizing against the corresponding cell control. WT = wild-type; ΔS = S1/S2 spike mutant.

These data show that acetylcysteine and bromelain alone do not induce SARS-CoV-2 inactivation (of either virus strain), but that these molecules have inactivating potential when used in combination, evidenced by the dose-dependent results from BromAc.

### Example 5 - Effect of BromAc on the Interaction of SARS-CoV-2 and Host Cells

SARS-CoV-2 binds to ACE-2 and NRP-1 receptors on human cells, and this is thought to be the mechanism via which internalisation occurs. The inventors have performed some preliminary experiments to assess whether BromAc may downregulate expression of NRP-1 and ACE-2 receptors.

In these experiments, ACE-2 and NRP-1 receptors were expressed in Vero and breast cells (MDA-MB-231) and were exposed to bromelain or acetylcysteine alone at varying concentrations and then combination. Specifically, Vero and MDA-MB-231 cells were treated with various concentrations of bromelain and acetylcysteine for 24 hours. The cells were then lysed by RIPA buffer supplemented with protease inhibitor. Protein concentration was determined using the BCA assay as per manufacturer’s instructions ((Pierce™ BCA Protein Assay Kit; Cat# 23225). 30 µg protein was then incubated at 95° C. in Laemmli loading buffer containing 10% DTT (Bio-Rad) for 5 minutes. Electrophoresis was conducted at 80V for 2 hour and proteins were transferred to PVDF membranes at 85V for 1 hour. Membranes were blocked in 5% skim milk in phosphate-buffered saline containing 0.05% Tween 20 (PBST) and then incubated with primary antibodies diluted in 5% bovine serum albumin in PBST overnight at 4° C. [Anti-Neuropilin-1 (1:1000, Cell signalling #3725) acetylcysteine and Anti-ACE2 Antibody (1:200, Santa Cruz Biotechnology# sc-390851)]. Membranes were then washed 5x using PBST and incubated with secondary antibody in PBST for 1 hour at room temperature. After five washes, proteins were visualized using SuperSignal™ Western Blot Enhancer (ThermoFisher Scientific, Cat#: 46641).

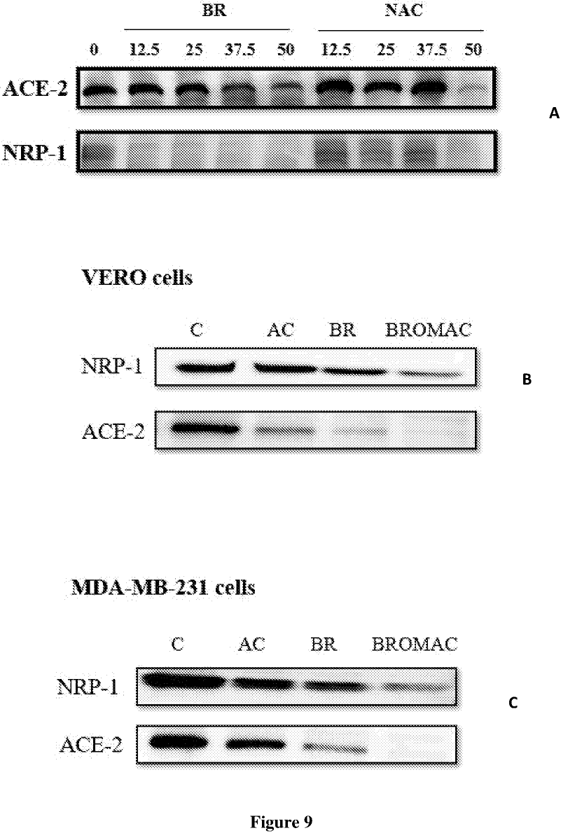

The inventors found that BromAc suppresses the protein expression of NRP-1 and ACE-2 receptors, but that Bromelain or Acetylcysteine do not. The results of the inventors’ experiments are shown in FIG. 9. In FIG. 9A, VERO Cells were treated for 24 hours with varying concentrations of bromelain or acetylcysteine. In FIGS. 9B and 9C, VERO (B) and MDA-MB-231 (C) cells were treated for 24 hours with 10 mM acetylcysteine (AC), 10 µg/mL bromelain (BR) or BromAc (BROMAC) and compared with a control (C). The downregulation of the host receptors may be another mechanism by which BromAc prevents or limits infection from SARS-CoV-2 (reduced receptors, reduced infection ability) in addition to its direct antiviral effects.

### Example 6 - Gel Electrophoresis Experiments on Ebola Spike and Envelope Proteins

Experiments similar to those described above in Example 1 were conducted to demonstrate that combinations of bromelain and acetylcysteine (NAC) cause ebolavirus spike proteins to disintegrate. In these experiments, recombinant spike proteins were treated at a range of concentrations of single agents and BromAc (i.e. bromelain and acetylcysteine in combination), with the resultant products being analysed using gel electrophoresis.

The spike proteins of two ebolavirus were tested. Ebola virus EBOV (subtype Bundibugoyo, strain Uganda 2007) GP1/Glycoprotein and Ebola virus EBOV (subtype Zaire, H.sapiens-wt/GIN/2014/Kissidougou-C15) Glycoprotein / GP.

The spike protein was reconstituted in sterile distilled water according to the manufacturer’s instructions and aliquots were frozen at -20° C. Bromelain and Acetylcysteine stock solutions were made in in Milli-Q water. Spike protein 5 µg was placed in micro-centrifuge tubes and 5 µg/ml, 10 µg/ml, 20 µg/ml, 25 µg/ml, 50 µg/ml and 100 µg/ml Bromelain, 20 mg/ml Acetylcysteine or a combination of both (i.e. BromAc) was added. The total reaction volume was 15 µL per tube. The control contained no Bromelain or Acetylcysteine.

All tubes were incubated at 37° C. for 30 min, after which 5 µl of sample buffer was added into each tube. SDS-Page precast gel from Bio-Rad was used for running the gel. Each well was loaded with 20 µL of each processed sample described above. Protein electrophoresis was performed in running buffer at 100 w for 1 hr. The gels were then immersed in Coomassie blue dye solution and gently shaken for 2 hr, after which the excess stain was removed by washing at room temperature.

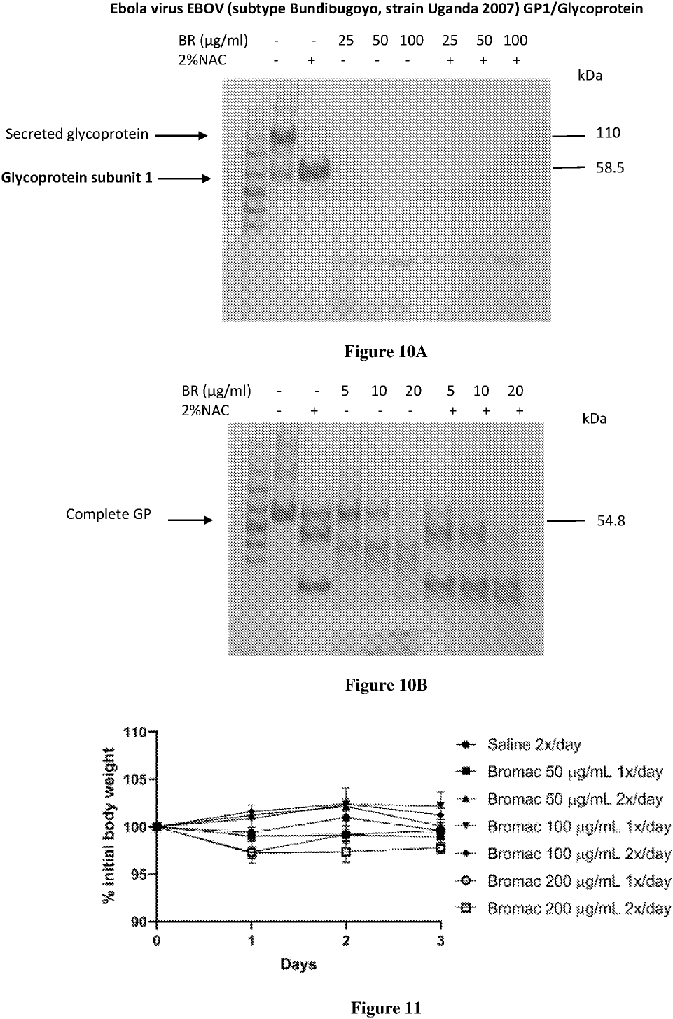

The results for Ebola virus subtype Bundibugoyo, strain Uganda 2007 GP1/Glycoprotein are shown in FIG. 10A. The recombinant GP1 protein is 53.5 kDa. As can be seen, treatment with Bromelain at such high concentrations (starting at 25 µg/ml) completely degraded the GP1 protein, whilst treatment with acetylcysteine only did not. In light of these data, the concentrations of bromelain tested in the BromAc used in the subsequent experiments with the other ebolavirus (described below) were therefore lowered.

The results for Ebola virus subtype Zaire, H.sapiens-wt/GIN/2014/Kissidougou-C15) Glycoprotein / GP are shown in FIG. 10B. The recombinant GP protein is 54.8 kDa. At 20 µg/ml Bromelain concentration, the glycoprotein was degraded. Treatment with the combination of 5 and 10 µg/ml Bromelain with Acetylcysteine was more effective than single agents, thus demonstrating a synergy similar to that described above in relation to the SARS-CoV-2 virus spike and envelope proteins.

### Example 7 - Safety Evaluation of Nasal Spray of BromAc in a Mouse Model

A tolerability study was carried out to investigate how well the mice tolerate the intranasal delivery of BromAc. A total of 126 C57BL/6 mice at 8 weeks of age were intranasally administered with 30 µL of solution at the concentrations and frequencies specified below:
1) 50 µg/mL bromelain with 20 mg/mL acetylcysteine (Low dose)
   a. Once daily (n=18)
   b. Twice daily (n=18)
2) 100 µg/mL bromelain with 20 mg/mL acetylcysteine (Medium dose)
   a. Once daily (n=18)
   b. Twice daily (n=18)
3) 200 µg/mL bromelain with 20 mg/mL acetylcysteine (High dose)
   a. Once daily (n=18)
   b. Twice daily (n=18)
4) Sterile saline solution (Vehicle control)
   a. Twice daily (n=18)

Mice were weighed daily and their clinical scores monitored. At 1, 3 and 5 days following administration of the initial intranasal dosages (n=42 for each endpoint), mice were euthanised. The mice appear to show no detrimental effects to the drugs based on body weight measurements (FIG. 11) and clinical scores. Following the initial intranasal dose, one mouse that received the high dose once daily displayed laboured breathing in the several hours post-administration, however this mouse recovered overnight and showed no significant changes to weight or clinical scores following this.

Following euthanasia of the mice and post-mortem examination and tissue harvest, the presence of a small dark spot on the liver of one mouse that received the medium dose once daily was observed, and one mouse that received the high dose once daily. It is likely that this was previously present prior to drug administration, as the cohort that received the medium and high doses twice daily showed no gross pathological changes to tissue morphology.

By comparison with control mice, treatment of mice with Brom/Ac 0.05, 0.1 or 0.2 mg/20 mg / mL did not show histological alteration in livers and kidneys in drug-treated mice, with no significant difference in lung histology between vehicle and treated groups.

### Example 8 - Aerosolization of Formulations Containing Bromelain and NAC

The inventors commissioned preliminary studies on the potential aerosolization of formulations containing NAC and bromelain. The following formulations were prepared in saline (0.9% w/v) and kept at -20° C. prior to analysis:
1. Control: Saline (sodium chloride 0.9% w/v)
2. Low Concentration of NAC (8 mg/mL)
3. High Concentration of NAC (60 mg/mL)
4. BR at 50 µg/mL
5. Combination 1: Low NAC (8 mg/mL) + BR (50 µg/mL)
6. Combination 2: High NAC (60 mg/mL) + BR (50 µg/mL)

The particle size distributions (PSD) of these formulations were determined. The formulations were aerosolized using PARI TurboBOY SX compressor, combined with the Pari LC Sprint nebulizer (PARI GmbH, USA). The nebulizer was connected to a USP induction port (throat), and particle size was measured at flow rate of 15 L/min in Spraytec particle sizer (Malvern Instruments, Malvern, UK). Measurements were performed in triplicate and the results are expressed as D10, D50 and D90, indicating the particle diameter at 10, 50 and 90% in the cumulative distribution.

All formulations were successfully nebulized and similar particle size distributions were observed for all formulations. The D50 of all formulations was smaller than 5 µm, which means that all formulations are deemed suitable for aerosol delivery to the lungs. To the best of the inventors’ knowledge, no one has ever nebulised bromelain for administration into the airway before.

As described herein, the present invention provides method for the prophylaxis or treatment of a viral infection in a patient. Embodiments of the present invention provide a number of advantages over existing therapies, some of which are described above.

It will be understood to persons skilled in the art of the invention that many modifications may be made without departing from the spirit and scope of the invention. All such modifications are intended to fall within the scope of the following claims.

It will be also understood that while the preceding description refers to specific forms of pharmaceutical compositions and methods of treatment, such detail is provided for illustrative purposes only and is not intended to limit the scope of the present invention in any way.

It is to be understood that any prior art publication referred to herein does not constitute an admission that the publication forms part of the common general knowledge in the art.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1-32. (canceled)

33. A method for the prophylaxis or treatment of a viral infection in a patient, the method comprising administering to the patient a therapeutically effective amount of a combination of a glycoprotein affecting protease and a disulphide bond breaking agent.

34. The method of claim 33, wherein the glycoprotein affecting protease is effective to hydrolyse glycosidic bonds of glycoproteins in the virus.

35. The method of claim 33, wherein the glycoprotein affecting protease is a cysteine protease.

36. The method of claim 33, wherein the glycoprotein affecting protease is bromelain.

37. The method of claim 33, wherein the disulphide bond breaking agent is acetylcysteine (NAC).

38. The method of claim 33, wherein the combination is administered into the lungs of the patient.

39. The method of claim 38, wherein the combination is nebulized before administration.

40. The method of claim 33, wherein the combination is nasally administered to the patient.

41. The method of claim 33, wherein the combination is administered to the patient immediately upon the patient becoming symptomatic.

42. The method of claim 33, wherein the combination is administered to the patient as a prophylactic.

43. The method of claim 33, wherein one or more additional therapeutic agents selected from the group consisting of antivirals, antibacterial agents and antiproteases are co-administered to the patient with the combination.

44. The method of claim 33, wherein the glycoprotein affecting protease, disulphide bond breaking agent and, optionally, any other additional therapeutic agent(s), are administered to the patient simultaneously, separately or sequentially.

45. The method of claim 33, wherein the viral infection is a viral respiratory disease.

46. The method of claim 33, wherein the viral infection is COVID-19 or Ebola virus disease.

47. A method for rendering a virus non-infective, the method comprising contacting the virus with a combination of a glycoprotein affecting protease and a disulphide bond breaking agent.

48. The method of claim 47, wherein the virus is a coronavirus such as severe acute respiratory syndrome coronavirus 2 (SARS-CoV2) or an ebolavirus.

49. The method of claim 47, wherein the virus is contacted with the combination of the glycoprotein digesting protease and disulphide bond breaking agent by spraying the combination onto the virus.

50. The method of claim 49, wherein the combination is sprayed into a patient using a nasal spray, throat spray or intra-tracheal spray.

51. The method of claim 49, wherein the combination is sprayed into the patient immediately upon the patient becoming symptomatic.

52. A method for preventing disease progression in a patient infected by a virus, the method comprising administering to the patient a therapeutically effective amount of a combination of a glycoprotein affecting protease and a disulphide bond breaking agent.
